(19) United States
(12) Patent Application Publication
(10) Pub. No.: US 2021/0244402 A1
(43) Pub. Date: Aug. 12, 2021

(54) Orthopedic Soft Knotless Anchor With Sheath Maintained in Place by a Closed Link

(71) Applicant: Dean Leffler, Nashville, TN (US)

(72) Inventor: Dean Leffler, Nashville, TN (US)

(21) Appl. No.: 17/167,869

(22) Filed: Feb. 4, 2021

Related U.S. Application Data
(60) Provisional application No. 62/971,854, filed on Feb. 7, 2020.

(51) Int. Cl. A61B 17/04

(52) U.S. Cl. CPC A61B 17/0401; A61F 2002/0852; A61B 2017/0445

(57) ABSTRACT

An all-suture anchor including an anchor suture having a closed loop at one end thereof; and a sheath positioned around at least a portion of the closed loop such that closed loop holds the sheath in a pre-defined location on the all-suture anchor. In this manner, the need for a knot at one end of the suture to hold the sheath in place is eliminated and the all-suture anchor is easier for surgeons to use as the knot is not interfering with the passage of repair sutures through the bone hole.

### CROSS-REFERENCE TO RELATED APPLICATIONS

This is a Non-Provisional Application claiming the benefit of co-pending Provisional Application No. 62/971,854 filed Feb. 7, 2020.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to a soft knotless anchor used for surgical procedures, namely orthopedic procedures.

### Background of the Invention

The current array of shoulder repair anchors, techniques, and constructs vary greatly and offer many choices to optimize soft-tissue healing to bone. The widely used all-suture anchor designs represent an innovative option that allows for versatile delivery and a low-profile anchor construct that minimizes bone drilling and preserves native bone. All-suture anchors are made by a myriad of companies (e.g., Arthrex, Zimmer Biomet, etc.)

All suture anchors are used for various types of surgeries, including labrum repairs, requiring soft tissue to be connected to bone. The all-suture anchors are configured, like rigid anchors, to insert into and remain in a hole in bone (e.g., a drilled hole).

It would be advantageous to develop an improvement to the manner in which the sheath of the all-suture anchor is held in place in addition to creating a highly reproducible solution for a knotless device to work properly and as intended.

### SUMMARY OF THE INVENTION

One embodiment of the present invention is directed to an all-suture anchor comprising an anchor suture having a closed loop at one end thereof; and a sheath positioned around at least a portion of said closed loop, said closed loop holding said sheath in a pre-defined location on said all-suture anchor. In this manner, the need for a knot at one end of the suture to hold the sheath in place is eliminated.

Other variations, embodiments and features of the present invention will become evident from the following detailed description, drawings and claims.

### BRIEF DESCRIPTION OF THE DRAWINGS

FIG. 1 illustrates a prior version of an all-suture anchor with a knot to hold the sheath in place.

FIGS. 2A and 2B illustrate prior versions of an all-suture anchor with a knot to hold the sheath in place.

FIG. 3 illustrates a first view of an improved all-suture anchor with a closed loop to hold the sheath in place according to the embodiments of the present invention.

FIG. 4 illustrates a second view of an improved all-suture anchor with a closed loop to hold the sheath in place according to the embodiments of the present invention.

FIG. 5 illustrates the improved all-suture anchor in place within bone according to the embodiments of die present invention.

### DESCRIPTION OF THE INVENTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

In broadest form, the embodiments of the present invention comprise an anchor suture having a closed loop at one end thereof; and a sheath positioned around at least a portion of said closed loop, said closed loop holding said sheath in a pre-defined location on said all-suture anchor. The all-suture anchor is easier for surgeons to use and eliminates the need for a knot to hold the sheath in place. Removal of the knot in lieu of the closed loop allows the implant to function as intended. The knot design causes interference with the passage of the repair sutures (i.e., creating an unnecessary blockage on the bone hole near the sheath).

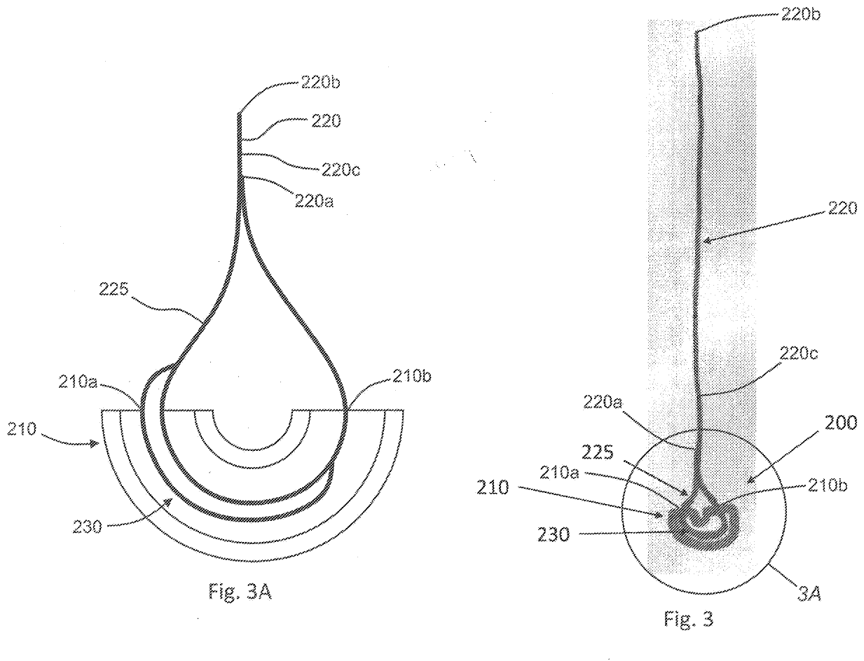

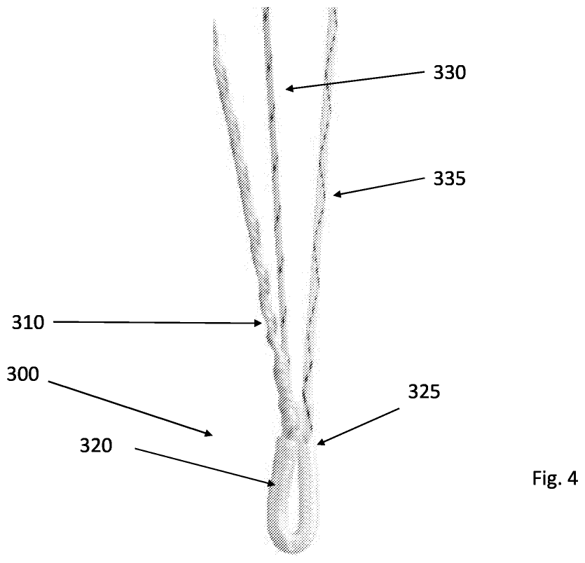

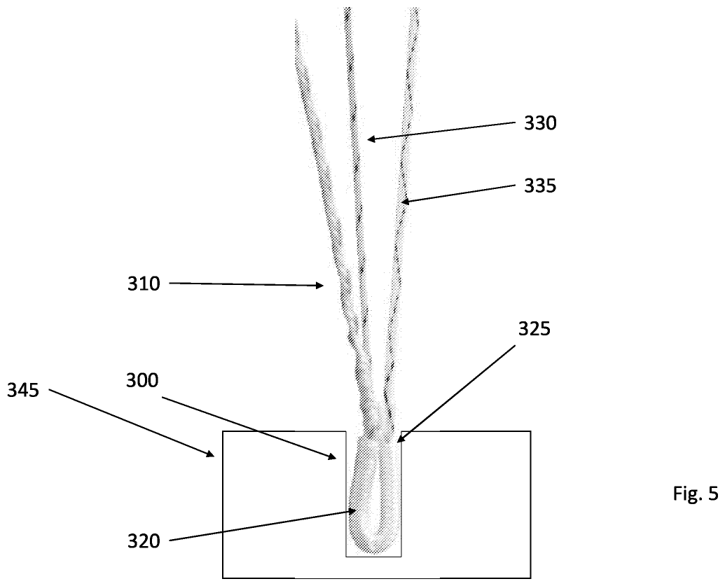

The embodiments of the present invention are applicable to various types of knotless all-suture anchors (e.g., Knotless Fibertak® Soft Anchor made by Arthex). Knotless all-suture anchors are used for soft tissue repair. Such devices allow for soft tissue repair to bone without the need for knot tying. This provides a particular advantage for arthroscopic repairs, where knot tying can be difficult and/or not desired. The knotless suture anchor is first inserted into bone. The repair suture is passed through soft tissue. The knotless suture anchor has a repair suture (See, 310 in FIGS. 4 and 5) and a shuttle suture (See, 330, 335 in FIGS. 4 and 5). The shuttle suture has a suture loop secured to the end of (See, 330 in FIGS. 4 and 5). This is intended for shuttling of the repair suture (See, 310 in FIGS. 4 and 5) through the locking mechanism (See, 230 FIG. 3) of the implant. A channel (locking mechanism) located near the tip of the anchor functions to capture the repair suture once shuttled through the channel with the shuttle suture. The shuttle suture is then discarded after shuttling the anchor suture through the channel. The repair suture is tensioned until desired fixation of tissue to bone is met. As the name suggests, the all-suture anchor relies on a soft suture sheath rather than a rigid component to secure the implant in bone.

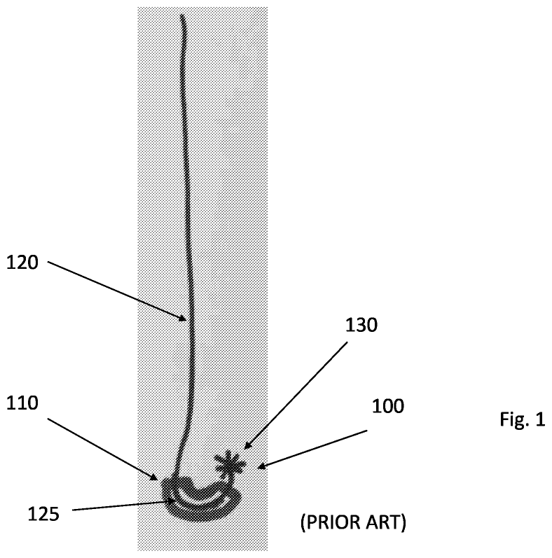

FIG. 1 shows a perspective view of a prior art all-suture anchor 100. As shown, sheath 110 encompasses an end portion of the anchor suture 120. Knot 130 prevents the sheath 110 from slipping off the end of the anchor suture 120. A locking mechanism 125 proximate the anchor suture 120 is encompassed by the sheath 110 as well.

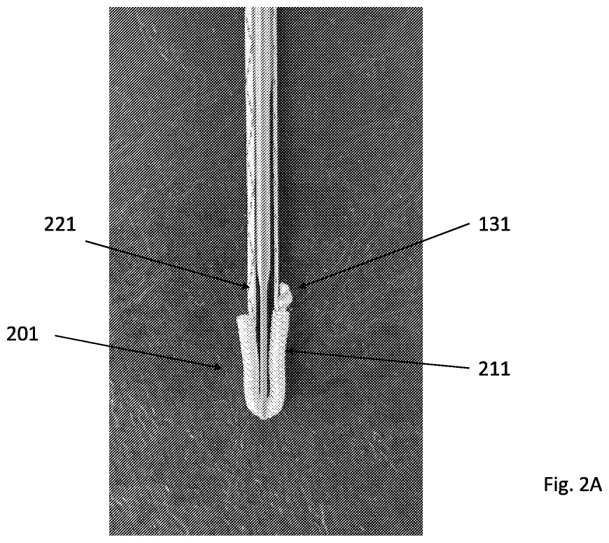

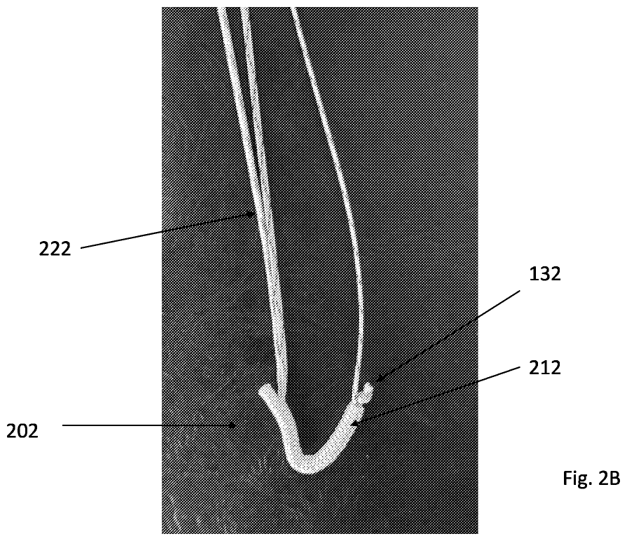

FIG. 2 shows a perspective view of another prior art all-suture anchor 150. As shown, sheath 160 encompasses an end portion of the anchor suture 170. Knot 180 prevents the sheath 160 from slipping off the end of the anchor suture 150.

FIG. 3 shows a perspective view of an all-suture anchor 200 according to the embodiments of the present invention. As shown, sheath 210 encompasses an end portion of the anchor suture 220. In this instance, the sheath 210 is held in place via closed loop 225 eliminating the need for the knot 130 shown in FIG. 1 (and FIGS. 2A and 2B). Accordingly, closed loop 225 prevents the sheath 220 from slipping off the end of the anchor suture 220. A locking mechanism 230 is encompassed by the sheath 210 as well. FIGS. 2A and 2B show similar all-suture anchors 201, 202 with a knot 131, 132, respectively, holding the sheaths 211, 212, respectively, in place on anchor sutures 221, 222.

FIG. 4 shows a perspective view of an all-suture anchor 300 according to the embodiments of the present invention. As shown, sheath 310 encompasses an end portion of the anchor suture 320 with sheath 310 is held in place via closed loop 325. A shuttle suture having a rounded portion 330 and flat portion 335 form part of the all-suture anchor 300.

FIG. 5 shows the all-suture anchor 300 inserted into bone 345.

Although the invention has been described in detail with reference to several embodiments, additional variations and modifications exist within the scope and spirit of the invention as described and defined in the following claims.

1. An all-suture anchor comprising:
(a) an anchor suture having a closed loop at one end thereof; and
(b) a sheath positioned around at least a portion of said closed loop, said closed loop holding said sheath in a pre-defined location on said all-suture anchor.

2. An all-suture anchor comprising:
(a) an anchor suture having a closed loop at one end thereof;
(b) a sheath positioned around at least a portion of said closed loop, said closed loop holding said sheath in a pre-defined location on said all-suture anchor; and
(c) a shuttle suture.

3. The all-suture anchor as defined in claim 2 in which said anchor suture has a first end, a second end and a connection area, said first end of said anchor suture being connected to said connection area of said anchor suture to form said closed loop.

4. The all-suture anchor as defined in claim 3 in which said sheath includes a body having first and second openings.

5. The all-suture anchor as defined in claim 4 in which said first end of said anchor suture extends through said first and second openings in said sheath.

6. The all-suture anchor as defined in claim 4 in which said shuttle suture has a flat portion.

7. A soft knotless anchor comprising:
(a) a sheath including a body having first and second openings; and
(b) an anchor suture connected to said sheath, said anchor suture having a first end, a second end and a connection area, said first end of said anchor suture extending through said first and second openings in said body of said sheath and then being connected to said connection area of said anchor suture to form a closed loop.

8. The soft knotless anchor as defined in claim 7 in which at least a portion of said closed loop is disposed within said body of said sheath.

9. The soft knotless anchor as defined in claim 7 further including a locking mechanism connected to said anchor suture and being disposed within said body of said sheath.

10. The soft knotless anchor as defined in claim 7 further including a shuttle suture connected to said anchor suture.

11. A soft knotless anchor comprising:
(a) a sheath including a body having first and second openings;
(b) an anchor suture connected to said sheath, said anchor suture having a first end, a second end and a connection area, said first end of said anchor suture extending through said first and second openings in said body of said sheath and then being connected to said anchor suture at said connection area to form a closed loop at least a portion of said closed loop being disposed within said body of said sheath;
(c) a locking mechanism connected to said anchor suture and disposed within said body of said sheath; and
(d) a shuttle suture connected to said anchor suture.
